(19) United States
(12) Patent Application Publication
(10) Pub. No.: US 2005/0054057 A1
(43) Pub. Date: Mar. 10, 2005

(54) Antibody specifically binding to sulfonylated protein and method for producing the same

(76) Inventors: Sang Won Kang, Seoul (KR); Young Ha Lee, Seoul (KR); Hyung Kee Kim, Seoul (KR); Kyung Yong Kim, Cheongju-city (KR); Jong Seo Lee, Suwon-city (KR); Soon Chun Kwon, Suwon-city (KR)

Correspondence Address: JONES DAY, 222 EAST 41ST ST, NEW YORK, NY, 10017, US

(21) Appl. No.: 10/932,688

(22) Filed: Sep. 2, 2004

(30) Foreign Application Priority Data
Sep. 4, 2003 (KR) 2003-61738

(51) Int. Cl. A61K 39/395; C12P 21/04; C07K 16/40

(52) U.S. Cl. 435/70.21; 530/388.25; 530/388.26

(57) ABSTRACT

Provided is a method for producing an antibody binding to a sulfonylated isoform of a protein; not to a non-sulfonylated isoform of the protein and other proteins, including: providing a peptide comprised of 7 to 15 amino acids derived from the protein and having a sulfonylated cysteine residue; inducing an antibody to the peptide; and isolating a population of antibodies reactive to the peptide.

### BACKGROUND OF THE INVENTION

This application claims the benefit of Korean Patent Application No. 2003-61738, filed on Sep. 4, 2003, in the Korean Intellectual Property Office, the disclosure of which is incorporated herein in its entirety by reference.

1. Field of the Invention

The present invention relates to a method for producing an antibody that specifically binds to a sulfonylated protein isoform.

2. Description of the Related Art

Reactive oxygen species (ROS) include a superoxide anion, hydrogen peroxide, and a hydroxyl radical. ROS can be generated from internal sources, such as mitochondria and enzyme systems (such as NADPH oxidase and 5-lipoxygenase). Further, ROS can be generated from external sources, such as ultraviolet light and y-ray. ROS can cuase oxidative damage to biomolecules, such as proteins, lipids, and DNAs. To protect cellular components against such oxidative damage, various antioxidant molecules exist, such as superoxide dismutase and peroxidase. Radical scavengers, an alternative, can be obtained from foods.

Nevertheless, cellular components continuously suffer from oxidative damage and are restored or destroyed. Such homeostasis is destroyed under various environments, such as stress or aging. When deterioration of such homeostasis sets in, diseases can be induced.

In proteins, a reactive cysteine residue is mainly oxidized under oxidative stress. The sufurhydryl group (Cys-SH) in cysteine is oxidized with hydrogen peroxide to sulfenic acid (Cys-SOH), sulfinic acid (Cys-SO₂H), and then sulfonic acid (Cys-SO₃H) sequentially. Protein oxidation under oxidative conditions, for example, peroxidations of glyceraldehyde dehydrogenase (GAPDH) and peroxiredoxins (Prxs) have been reported [Rabilloud T. etc., J. Biol. Chem. 277: 19396˜19401, 2002; Yang K. S etc., J. Biol. Chem. 277: 38029˜38036, 2002]. The reactive sites of the above two proteins are oxidized to form Cys-SO₂H and Cys-SO₃H, thus losing their enzymatic activities. Especially, peroxidation of peroxiredoxins was dectected in cells treated with tumor necrosis factors (TNFs) and occurs during a process of signal transductio. Recently, the fact that Cys-SO₂H of peroxiredoxin type I can be reversibly reduced to a sulfhydryl group in various cellular forms has been reported [Woo H. A. etc., Science, 300:653˜656, 2003]. Due to such peroxidation of cysteine residue, the protein may lose its fuction. Thus, a degree of oxidation of the cysteine residue can be indicative of changes of protein functions and healthy cells. Thus, detection of Cys-SO₂H and Cys-SO₃H in the oxidized protein is important since it can demonstrate that the cells are under oxidative stress and the functions of the cells have been changed due to a loss of activity of target proteins.

A method for producing a polyclonal antibody specifically binding to a conventional protein antigen is well known in the art. Further, a method for producing a monoclonal antibody having monospecificity to a specific protein antigen is well known in the art.

However, an antibody specifically binding to a sulfonylated protein has not been disclosed. Thus, the present inventors conducted vigorous research to obtain a method for producing an antibody specifically binding to a sulfonylated protein; not to a non-sulfonylated form of the protein and other proteins.

### SUMMARY OF THE INVENTION

The present invention provides a method for producing an antibody specifically binding to a sulfonylated protein, not to a non-sulfonylated form of the protein and other proteins.

The present invention also provides an antibody specifically binding to a sulfonylated protein produced using the above method.

According to an aspect of the present invention, there is provided a method for producing an antibody binding only to a sulfonylated isoform of a protein; not to a non-sulfonylated isoform of the protein and other proteins, comprising: providing a peptide comprised of 7 to 15 amino acids derived from the protein and having a sulfonylated cysteine residue; inducing an antibody to the peptide; and isolating a population of antibodies reactive to the peptide.

According to another aspect of the present invention, there is provided an antibody which specifically binds only to a sulfonylated isoform of a protein having a sulfonylated cysteine residue; not to a non-sulfonylated isoform of the protein and other proteins.

### BRIEF DESCRIPTION OF THE DRAWINGS

The above and other features and advantages of the present invention will become more apparent by describing in detail exemplary embodiments thereof with reference to the attached drawings in which:

FIG. 1 is a schematic view illustrating each active site of Prx I, Prx VI, and GAPDH;

FIG. 2A is a schematic view illustrating peptides 1 and 4;

FIG. 2B is a chromatogram for peptides 1 and 4;

FIGS. 2C and 2D are views illustrating results of a mass spectroscopy for peptides 1 and 4, respectively;

FIG. 3A is a schematic view illustrating peptides 2 and 5;

FIG. 3B is a chromatogram for peptides 2 and 5;

FIGS. 3C and 3D are views illustrating results of a mass spectroscopy for peptides 2 and 5, respectively;

FIG. 4A is a schematic view illustrating peptides 3 and 6;

FIG. 4B is a chromatogram for peptides 3 and 6;

FIGS. 4C and 4D are views illustrating results of mass spectroscopy for peptides 3 and 6, respectively;

FIG. 5A is a view illustrating results of western blotting of Prx I treated or not treated with H₂O₂, detected using HRP-anti SO₃-Prx I (upper portion) and HRP-anti-Prx I (lower portion);

FIG. 5B is a view illustrating results of western blotting of HeLa cell lysate not treated with H₂O₂ (lane 1), HeLa cell lysate treated with H₂O₂ (lane 2), Prx I in a reduced state (lane 3), and Prx I in an oxidized state (lane 4), detected using HRP-anti SO₃-Prx I (upper portion) and HRP-anti-Prx I (lower portion);

FIG. 6A is a view illustrating results of western blotting of Prx VI treated or not treated with H₂O₂, detected using HRP-anti SO₃-Prx VI (upper portion) and HRP-anti-Prx VI (lower portion);

FIG. 6B is a view illustrating results of western blotting of HeLa cell lysate not treated with H₂O₂ (lane 1), HeLa cell lysate treated with H₂O₂ (lane 2), Prx VI in a reduced state (lane 3), and Prx VI in an oxidized state (lane 4), detected using HRP-anti SO₃-Prx VI (upper portion) and HRP-anti-Prx VI (lower portion);

FIG. 7A is a view illustrating results of western blotting of GAPDH treated or not treated with H₂O₂, detected using HRP-anti SO₃-GAPDH (upper portion) and HRP-anti-GAPDH (lower portion);

FIG. 7B is a view illustrating results of western blotting of HeLa cell lysate not treated with H₂O₂ (lane 1), HeLa cell lysate treated with H₂O₂ (lane 2), GAPDH in a reduced state (lane 3), and GAPDH in an oxidized state (lane 4), detected using HRP-anti SO₃-GAPDH (upper portion) and HRP-anti-GAPDH (lower portion);

FIG. 8 is a view illustrating results of western blotting of HeLa cell lysate not treated with H₂O₂ (lane 1), HeLa cell lysate treated with H₂O₂ (lane 2), Prx I in a reduced state (lane 3), and Prx I in an oxidized state (lane 4), detected using HRP-anti SO₃-Prx I monoclonal antibody (clone 2A1)(upper portion) and HRP-anti-Prx I (lower portion);

FIG. 9 is a view illustrating results of western blotting of 20 ug of HeLa cell lysate not treated with H₂O₂ (lane 1), 20 ug of HeLa cell lysate treated with H₂O₂ (lane 2), 40 ug of HeLa cell lysate not treated with H₂O₂ (lane 3), and 40 ug of HeLa cell lysate treated with H₂O₂ (lane 4), detected using HRP-anti SO₃-Prx I monoclonal antibody (clone 10A1)(upper portion) and HRP-anti-Prx I (lower portion);

FIG. 10 is a view illustrating results of western blotting of 20 ug of HeLa cell lysate not treated with H₂O₂ (lane 1), 20 ug of HeLa cell lysate treated with H₂O₂ (lane 2), 40 ug of HeLa cell lysate not treated with H₂O₂ (lane 3), and 40 ug of HeLa cell lysate treated with H₂O₂ (lane 4), detected using HRP-anti SO₃-Prx VI monoclonal antibody (clone 2G5)(upper portion) and HRP-anti-Prx VI (lower portion); and

FIG. 11 is a view illustrating results of western blotting of 20 ug of HeLa cell lysate not treated with H₂O₂ (lane 1), 20 ug of HeLa cell lysate treated with H₂O₂ (lane 2), 40 ug of HeLa cell lysate not treated with H₂O₂ (lane 3), and 40 ug of HeLa cell lysate treated with H₂O₂ (lane 4), detected using HRP-anti SO₃-GAPDH monoclonal antibody (clone 4A1)(upper portion) and HRP-anti-GAPDH (lower portion).

### DETAILED DESCRIPTION OF THE INVENTION

According to an embodiment of the present invention, there is provided a method for producing an antibody binding only to a sulfonylated isoform of a protein; not to a non-sulfonylated isoform of the protein and other proteins, comprising:
providing a peptide comprised of 7 to 15 amino acids derived from the protein and having a sulfonylated cysteine residue;
inducing an antibody to the peptide; and
isolating a population of antibodies reactive to the peptide. As used herein, the term “protein” refers to any protein having a cysteine residue. Examples of the protein include glyceraldehyde-3-phosphate dehydrogenase (GAPDH) or peroxiredoxins (Prxs). Preferably, the protein may be Prx I (Genbank accession No. NM_{—}002574), Prx VI (Genbank accession No. NM_{—}004905), or GAPDH (Genbank accession No. J04038).

According to the embodiment of the present invention, in the providing a peptide comprised of 7 to 15 amino acids and having a sulfonylated cysteine residue, the sulfonylated peptide includes, but are not limited to, any one of peptides having amino acid sequences of SEQ ID Nos. 1, 2, and 3 in which the —SH group of a Cys residue is oxidized to a —SO₃H group, respectively. The sulfonylated peptide may be produced using conventional methods, for example, chemical synthesis and synthesis by enzymatic treatment. The chemical synthesis method includes a method of synthesizing a sulfonylated peptide by oxidizing a peptide sequence having a cysteine residue with an oxidizing agent, such as performic acid. The sulfonylated peptide may be preferably comprised of 7 to 15 amino acids, and more preferably, 8 to 12 amino acids. If the number of the amino acids is less than 7, an antibody specific to a sulfonyl group of the sulfonylated protein is not easily induced. If the number of the amino acids is more than 15, not only an antibody specific to a sulfonyl group of the sulfonylated protein but also an antibody specific to other sites of the sulfonylated protein is induced, and thus, the antibody specific to a sulfonyl group should be further examined and purified, which increases production costs. In this way, in the present embodiment, the use of the short peptide induces a high proportion of the antibody specifically binding to a sulfonyl group in a subject, thus allowing easy selection of the antibody specific to the sulfonylated isoform among all the proteins having the above peptide sequence.

In an embodiment of the present invention, the sulfonylated peptide is administered to a subject to induce an antibody to the sulfonylated peptide. A method of administration of the peptide and animals used in producing antibodies are well known in the art and are not specifically limited. Examples of the animals may include, but are not limited to, mice, rabbits, goats, and chickens. The sulfonylated peptide attached to a support protein, such as hemocyanin, and then combined with an adjuvant can be administered.

In an embodiment of the present invention, in the isolation of a population of antibodies which are reactive to the sulfonylated peptide, the antibody specifically binding to the sulfonylated peptide is collected from the animal immunized with the sulfonylated peptide. The process of collecting the antibody may include collecting a blood sample from the immunized animal and isolating and purifying the blood sample using a conventional method, such as salting-out, ion exchange chromatography, gel permeation chromatography, affinity chromatography, and ultra-filtration, but being not limited thereto. Since the short peptide is used as an antigen in the present embodiment, an antibody containing the peptide and specifically binding to a protein haivng a sulfonylated cysteine residue can be obtained in high purity, by isolating the antibody specifically binding to the peptide.

The method according to an embodiment of the present invention may further comprise screening an antibody unreactive to the non-sulfonylated protein isoform having a non-sulfonylated cysteine residue from the population of antibodies. The screening may be performed by removing an antibody binding to the non-sulfonylated protein isoform, and not to a sulfonylated protein isoform from the antibodies obtained in the previous process. The screening may be performed using column chromatography filled with, for example, peptides having amino acid sequences of SEQ ID Nos. 1, 2, and 3 or peptides conjugated with ligand materials.

The method according to an embodiment of the present invention may further comprise verifying that the collected antibodies specifically bind to the protein isoform having a sulfonylated cysteine residue; not to other proteins by performing analysis. The verification can be performed in vitro, in vivo, or their combination using various conventional analytical methods. An example of the analytical method may include western blotting using, for example, a secondary antibody conjugated with an enzyme capable of producing a color (for example, horse-radish peroxidase (HRP)) (HRP-anti-rabbit immunoglobulin-antidoby) and the antibody specifically binding to the protein isoform having a sulfonylated cysteine residue.

According to another embodiment of the present invention, there is provided an antibody which specifically binds to a sulfonylated protein isoform having a sulfonylated cysteine residue; not to a non-sulfonylated isoform of the protein and other proteins. Examples of the antibody include, but are not limited to, an antibody which specifically binds to a sulfonylated Prx I isoform having a sulfonylated Cys 52, not to Prx I having a non-sulfonylated Cys 52 and other proteins, an antibody which specifically binds to a sulfonylated Prx VI isoform having a sulfonylated Cys 47, not to Prx VI having a non-sulfonylated Cys 47 and other proteins, and an antibody which specifically binds to a sulfonylated GAPDH isoform having a sulfonylated Cys 152, not to GAPDH having a non-sulfonylated Cys 152 and other proteins. The antibody according to the present embodiment may be produced using the method for producing an antibody specifically binding to a sulfonylated protein isoform according to the previous embodiment of the present invention.

Hereinafter, the present invention will be described in more detail with reference to the following examples. However, these examples are given for the purpose of illustration and are not intended to limit the scope of the invention.

### EXAMPLE

In the EXAMPLE, an antibody specifically binding to sulfonylated peroxiredoxin type I (Prx I), sulfonylated peroxiredoxin type VI (Prx VI), or sulfonylated glyceraldehyde-3-phosphate dehydrogenase (GAPDH) (hereinafter, also referred to as SO₃-protein antibodies) was developed.

First, a peptide comprising an active-site cysteine residue (a reduced state of peptide) is synthesized, and then oxidized by reacting with performic acid to obtain a sulfonylated peptide. The oxidized peptide was purified by C₁₈-reverse phase HPLC and analyzed using MALDI-TOF mass spectroscopy. The results of mass spectroscopy show that only Cys-SH in the peptide was oxidized to Cys-SO₃H to provide the sulfonylated peptide. The sulfonylated peptide was conjugated to a support protein, keyhole limpet hemocyanin (KLH), mixed with an adjuvant, and then injected subcutaneously into a rabbit. After a series of boost injections, a blood sample was collected from the rabbit and purified using salting-out to obtain the antibodies. Optionally, an antibody specific to the reduced state of the peptide was further removed from the obtained antibodies by passing the obtained antibodies through a column filled with beads to which the reduced state of the peptide was attached.

Further, monoclonal antibodies specifically binding to sulfonylated Prx I, sulfonylated Prx VI, and sulfonylated GAPDH, respectively, were produced. First, cells producing the antibody specifically binding to the above proteins were respectively isolated from spleen of a mouse which could produce the antibody, and fused to myeloma cells to produce hybridoma cells. Cells producing the antibodies specifically binding to the proteins were selected from the hybridoma cells.

### Example 1

### Synthesis of Sulfonylated Peptide

Active sites of Prx I (Genbank accession No. NM_{—}002574), Prx VI (Genbank accession No. NM_{—}004905), and GAPDH (Genbank accession No. J04038) are Cys-52, Cys47, and Cys-152, respectively. FIG. 1 is a schematic view illustrating each active site of Prx I, Prx VI, and GAPDH. 47˜56 Prx I (peptide 1), 42˜51 Prx VI (peptide 2), and 145˜155 GAPDH (peptide 3) including the above respective active sites were synthesized by standard Merrifield Solid-Phase Peptide Synthesis (SPPS) using t-BOC.

The synthesized peptides were respectively purified to 90 to 95% purity using Vydac C18 5 μm column (250 mm×4.6 mm). Each purified peptide was analyzed using mass spectroscopy to confirm that the desired peptide was synthesized. 2 mg of each peptide in its reduced form were directly dissolved in 20 μl of performic acid produced by mixing formic acid and hydrogen peroxide in a volume ratio of 9:1, respectively, and cultured at 25° C. for 1 hour to oxidize the peptide. The reactant solution was placed on ice for 50 minutes and then dried in a Thermo Savant SPD 1010 Speed-Vac instrument for 15 minutes without heating. The dried peptide was dissolved in 200 μl of water to a concentration of 2 mg/ml. To analyze an oxidized state of the peptide, 10 μg of the oxidized peptide were applied to Vydac C18 5 μm column which had been pre-equilibrated with 0.1% trifluoroacetic acid (TFA) in water, and subjected to a linear gradient ranging from 0.1% TFA in water to 0.09% TFA in acetonitrile for 60 minutes.

As a result, main peaks corresponding to oxidized peptides of peptide 1, peptide 2, and peptide 3 (hereinafter, referred to as peptides 4, 5 and 6, respectively) were observed at 34 min, 28.5 min, and 17.5 min, respectively. Compounds of these peaks were analyzed using a Voyager-STR MALDI-TOF instrument equipped with a nitrogen laser.

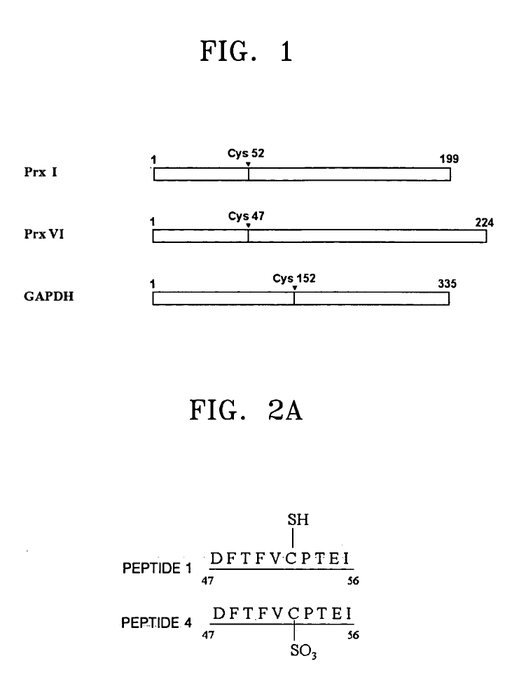

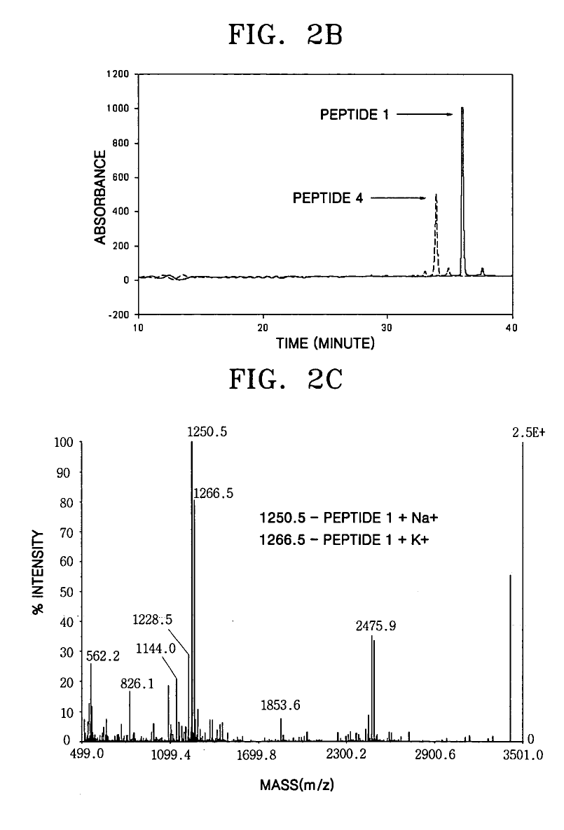

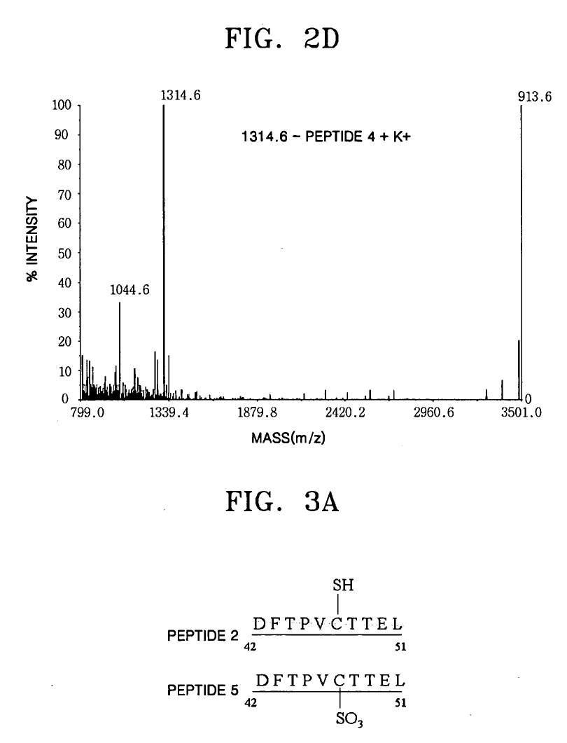

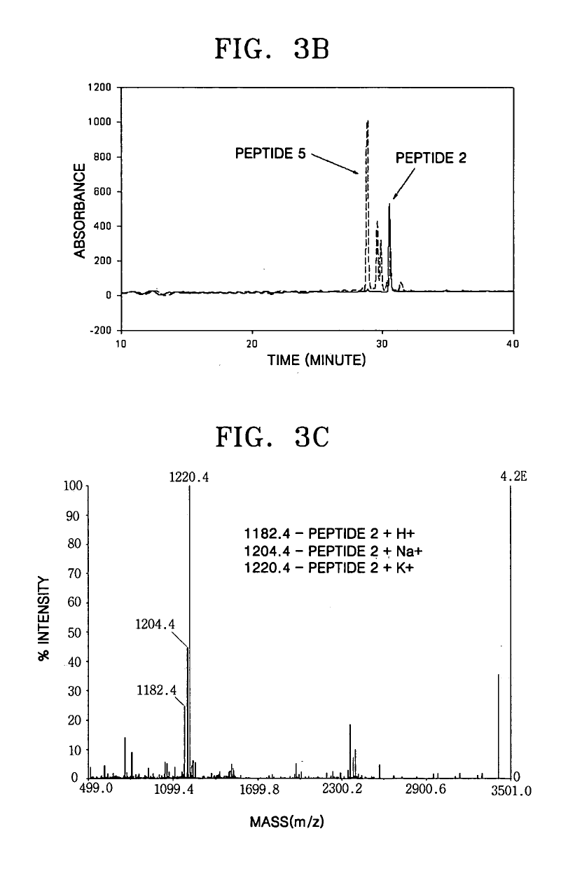

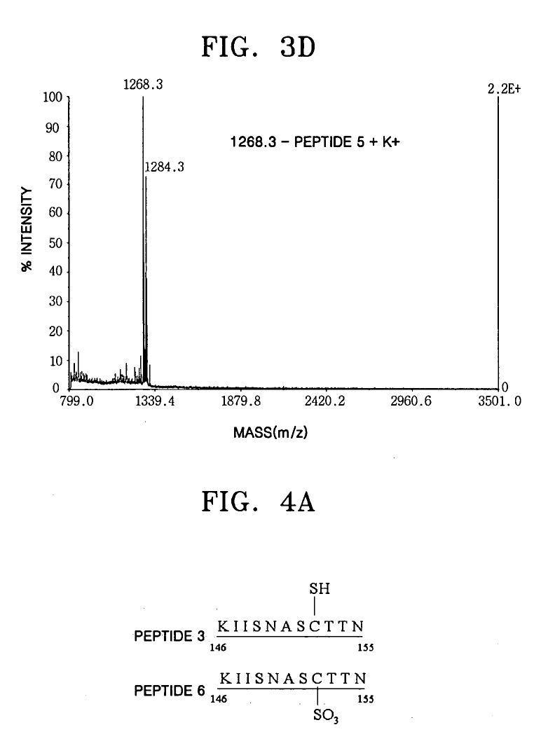

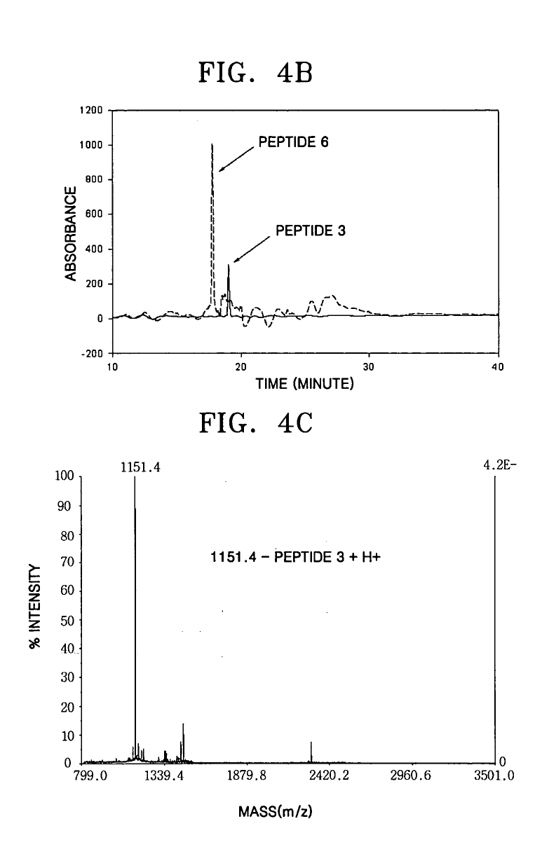

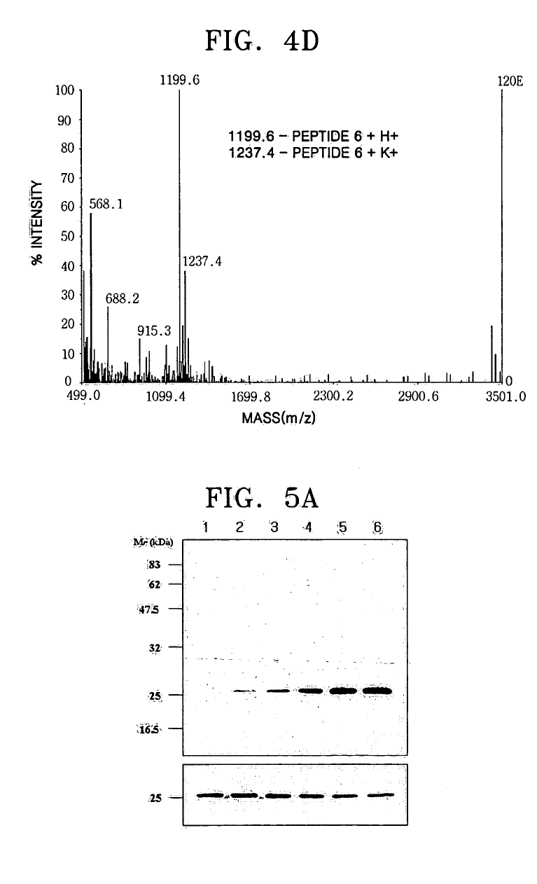

The results are shown in FIGS. 2A through 4D. FIG. 2A is a schematic view illustrating peptides 1 and 4. FIG. 2B is a chromatogram for peptides 1 and 4. FIGS. 2C and 2D are views illustrating results of a mass spectroscopy for peptides 1 and 4, respectively. FIG. 3A is a schematic view illustrating peptides 2 and 5. FIG. 3B is a chromatogram for peptides 2 and 5. FIGS. 3C and 3D are views illustrating results of a mass spectroscopy for peptides 2 and 5, respectively. FIG. 4A is a schematic view illustrating peptides 3 and 6. FIG. 4B is a chromatogram for peptides 3 and 6. FIGS. 4C and 4D are views illustrating results of mass spectroscopy for peptides 3 and 6, respectively. As illustrated in FIGS. 2A through 4D, it was confirmd that three oxygen atoms were attached to each peptide to form a sulfonylated peptide having Cys-SO₃H.

### Example 2

### Production and Purification of Antibody

2 mg of each sulfonylated peptide obtained from Example 1, i.e., peptides 4 and 6, were attached to 10 mg of keyhole limpet hemocyanin (KLH) (Pierce Chemical Co.) at room temperature overnight in the presence of 7 mM glutaraldehyde in a 0.1 M sodium phosphate buffer solution (pH 7.0). The obtained peptide-KLH conjugates were stored at −20° C. for ready-to-use. The conjugates were used as a mixture with Complete Freund's adjuvant for a primary injection and as a mixture with incomplete Freund's adjuvant for a boost injection. Rabbits were inoculated with each conjugate once every four weeks. 1 mg of the peptide for the primary injection and 500 μg of the peptide for the boost injection were subcutaneously injected at various locations on the rabbits. One week after boosting, 20 to 60 ml of antisera were collected and IgG fractions were precipitated with 50% (w/v) ammonium sulfate for a partial purification.

Since crude antisera substantially monospecific to the sulfonylated proteins were produced in many antibody-producing batches, it was not required an dditional reverse purification using beads to which a reduced state of the peptide is attached. However, when the antisera recognized a natural protein in a reduced state, the reduced state of peptide, i.e., peptide 1, 2, or 3, was attached to Affigel-10 chromatography beads (Bio-rad), a column was filled with the beads, and then the crude antisera were subjected to a reverse purification. The reverse purified antibody exhibited monospecificity to the sulfonylated proteins. Thus, it was confirmed from the results that most antisera are composed of the antibody specific to the sulfonylated protein, i.e, the SO₃-protein antibody.

### Example 3

### Confirmation of Reactivity of Polyclonal Antibody Specific to Sulfonylated Protein

In this Example, polyclonal antibodies specific to the sulfonylated proteins obtained from Example 2 were examined for their reactivity with the proteins in a reduced state, the proteins in an oxidized state, and HeLa cell lysates.

Production of Oxidized Cell Lysate and Recombinant Protein

HeLa cells used in the Example were kept in Dulbecco's minimal essential medium (DMEM) supplemented with 10% bovine serum, glutamate, and antibiotics. Production of HeLa cells treated with H₂O₂ and oxidized recombinant proteins were known in the art [Yang K. S. et al., J. Biol. Chem. 277: 38029˜38036, 2002].

Production of HeLa cells treated with H₂O₂ and oxidized recombinant proteins will be described in brief as follows. HeLa cells (1×10⁶ cells) were treated 1 mM H₂O₂ in the presence of 10% bovine serum for 30 minutes, washed with cold PBS, and then lysated with 1 ml of a lysis buffer. The lysis buffer comprised 20 mM Hepes (pH 7.0), 1 mM EDTA, 25 mM 1-phosphoglycerate, 10% glycerol, 150 mM NaCl, 1% Triton X-100, 5 mM sodium fluoride, 1 mM sodium ortho-vanadate, 10 ng/ml microcystin, and protease inhibitor (5 μg/ml leupeptin, 5 μg/ml aprotinin, and 1 mM 4-(2-aminoethyl)-benzenesulfonyl fluoride hydrochloride (AEBSF)). The lysate (30 μg of total protein) was used in immunoblot analysis.

Recombinant Prx proteins, Prx I to IV (each 10 μg) were incubated in 200 μl of a reactant mixture containing 1 mM H₂O₂ in 50 mM Hepes-NaOH buffer (pH 7.0) containing 200 mM NADPH, 2.5 mM hTrx, 46 nM rTrxR, and 1 mM EDTA. H₂O₂ was added to initiate the reaction and the reaction was continued at 30° C. for 30 minutes. In the reaction, all Prx proteins were completely oxidized to the sulfonylated forms, which was evidenced from the fact that spots of the proteins had moved to an acidic zone in a two-dimensional gel electrophoresis. A control reaction was performed without H₂O₂.

Recombinant GAPDH and Prx VI proteins (each 10 μg) were incubated in 200 μl of a reactant mixture containing 1 mM H₂O₂ in a 50 mM Hepes-NaOH buffer (pH 7.0) containing 5 mM dithiotratol (DTT) and 1 mM EDTA. H₂O₂ was added to initiate the reaction and the reaction was continued at 30° C. for 10 minutes. In the reaction, all proteins were completely oxidized to the sulfonylated forms, which was evidenced from the fact that spots of the proteins had moved to an acidic zone in a two-dimensional gel electrophoresis. A control reaction was performed without H₂O₂.

Western Blotting Analysis

The above proteins were isolated using SDS-PAGE and electrophoretically transferred to a nitrocellulose membrane. The membrane was blocked with skim milk, and then incubated with a rabbit's antibody specific to the sulfonylated peptides. Immune complexes were detected using an HRP-conjugated secondary antibody and an enhanced chemiluminescence reagent (available from Pierce).

After the detection, to confirm that an equal amount of proteins were loaded in each lane, the above membrane was treated with 62.5 mM Tris-HCl (pH 6.8) buffer containing 2% SDS and 100 mM β-mercaptoethanol at 65° C. for 30 minutes to separate the attached antibodies from the membrane. Next, the membrane was washed with water, and then tris-buffered saline containing 0.1% Tween-20. The membrane was analyzed by western blotting in the same manner as above using the rabbit antibody that can recognize both oxidized and reduced isoforms of the proteins.

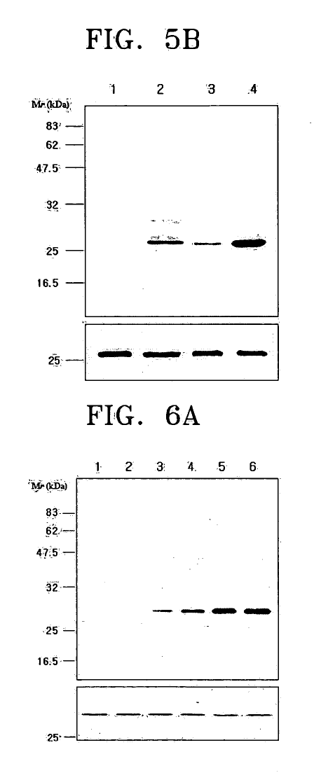

The results are shown in FIGS. 5A through 7B. The upper portion of FIG. 5A is a view illustrating results of western blotting of Prx I treated with H₂O₂ and Prx I not treated with H₂O₂, detected using HRP-anti SO₃-Prx I, after an electrophoresis of mixtures of the H₂O₂-treated Prx I and the not treated Prx I being loaded in a constant total amount as shown in Table 1. The lower portion of FIG. 5A is a view illustrating results of performing again western blotting of the above membrane, detected using HRP-anti-Prx I, to confirm that an equal total amount of Prx Is was loaded in each lane. The upper portion of FIG. 5B is a view illustrating results of western blotting of HeLa cell lysate not treated with H₂O₂ (lane 1), HeLa cell lysate treated with H₂O₂ (lane 2), Prx I in a reduced state (lane 3), and Prx I in an oxidized state (lane 4), detected using HRP-anti SO₃-Prx I. The lower portion of FIG. 5B is a view illustrating results of performing again western blotting of the above membrane, detected using HRP-anti-Prx I, to confirm that the same total amount of Prx Is was loaded in each lane.

TABLE 1
Loading amount in each lane (unit: ng)

| H₂O₂ | Lane 1 | Lane 2 | Lane 3 | Lane 4 | Lane 5 | Lane 6 |
|---|---|---|---|---|---|---|
| Treated Prx | 0 | 2.5 | 10 | 25 | 40 | 50 |
| Non-treated | 50 | 47.5 | 40 | 25 | 10 | 0 |
| Prx | | | | | | |

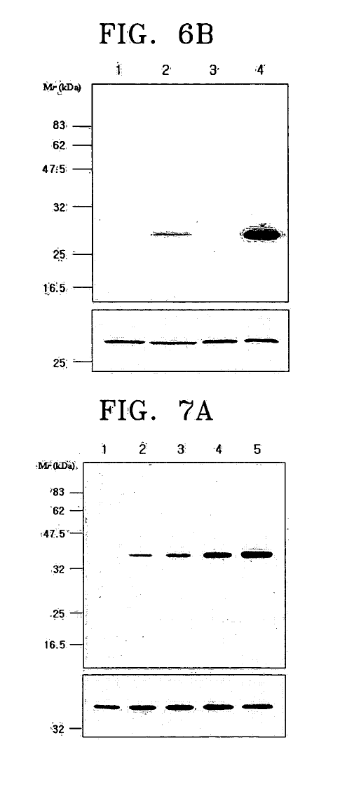

The upper portion of FIG. 6A is a view illustrating the results of western blotting of Prx VI treated with H₂O₂ and Prx VI not treated with H₂O₂, detected using HRP-anti SO₃-Prx VI, after an electrophoresis of mixtures of the H₂O₂-treated Prx VI and the not treated Prx VI being loaded in a constant total amount as shown in Table 1. The lower portion of FIG. 6A is a view illustrating results of performing again western blotting of the above membrane, detected using HRP-anti-Prx VI, to confirm that the same total amount of Prx Vis was loaded in each lane. The upper portion of FIG. 6B is a view illustrating results of western blotting of HeLa cell lysate not treated with H₂O₂ (lane 1), HeLa cell lysate treated with H₂O₂ (lane 2), Prx VI in a reduced state (lane 3), and Prx VI in an oxidized state (lane 4), detected using HRP-anti SO₃-Prx VI. The lower portion of FIG. 6B is a view illustrating results of performing again western blotting of the above membrane, detected using HRP-anti-Prx VI, to confirm that the same total amount of Prx Vls was loaded in each lane.

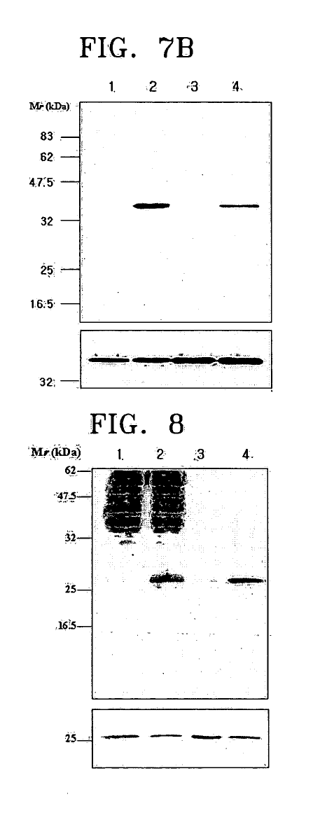

The upper portion of FIG. 7A is a view illustrating results of western blotting of GAPDH treated with H₂O₂ and GAPDH not treated with H₂O₂, detected using HRP-anti SO₃-GAPDH, after an electrophoresis of mixtures of the H₂O₂-treated GAPDH and the not treated GAPDH being loaded in a constant total amount as shown in Table 2. The lower portion of FIG. 7A is a view illustrating results of performing again western blotting of the above membrane, detected using HRP-anti-GAPDH, to confirm that the same total amount of GAPDHs was loaded in each lane. The upper portion of FIG. 7B is a view illustrating results of western blotting of HeLa cell lysate not treated with H₂O₂ (lane 1), HeLa cell lysate treated with H₂O₂ (lane 2), GAPDH in a reduced state (lane 3), and GAPDH in an oxidized state (lane 4), detected using HRP-anti SO₃-GAPDH. The lower portion of FIG. 7B is a view illustrating results of performing again western blotting of the above membrane, detected using HRP-anti-GAPDH, to confirm that the same total amount of GAPDHs was loaded in each lane.

TABLE 2
Loading amount in each lane (unit: ng)

| H₂O₂ | Lane 1 | Lane 2 | Lane 3 | Lane 4 | Lane 5 |
|---|---|---|---|---|---|
| Treated GAPDH | 0 | 12.5 | 25 | 50 | 100 |
| Not-treated | 100 | 87.5 | 75 | 50 | 0 |
| GAPDH | | | | | |

From the above results, it was confirmed that the antibody produced using a method according to an embodiment of the present invention specifically binds to the sulfonylated protein exhibiting low cross-reactivity.

### Example 4

### Production of Monoclonal Antibody Specific to Sulfonylated Protein

In this Example, a monoclonal antibody specific to each of the sulfonylated peptides 4 to 6 synthesized in Example 1 was produced and confirmed for its properties.

(1) Abbreviation and ELISA Test Procedure

Abbreviation

Base medium: DMEM (500 ml)
10% FBS medium: FBS 50 ml, penicillin/streptomycin 5 ml, DMEM 500 ml
1× HAT medium: 1 vial of HAT complement, FBS 100 ml, penicillin/streptomycin 5 ml, DMEM 500 ml
HT medium: 1 vial of HT complement, FBS 100 ml, penicillin/streptomycin 5 ml, DMEM 500 ml
freezing medium: FBS 25 ml, DMSO 5 ml, DMEM 20 ml
RBC lysate-buffer: NH₄Cl 0.83 g, 1M HEPES buffer 1 ml, distilled water up to a total of 100 ml
ELISA coating buffer: 0.2M NaH₂CO₃ 170 ml, 0.2M Na₂HCO₃ 80 ml, distilled water 250 ml
citrate buffer: citrate 9.45 g, Na₂HPO₄.12H₂O 19.6977 g, distilled water up to a total of 1 L
10× TBS-T (pH 7.4): 100 mM Tris 12.11 g, 1.5M NaCl 87.66 g, Tween 20 10 ml, distilled water up to a total of 1 L
ELISA chromophore: citrate buffer 10 ml, OPD 4 mg, 30% H₂O₂ 5 μl
1× PBS (pH 7.2): NaCl 8 g, KCl 0.2 g, Na₂HPO₄ 1.44 g, KH₂PO₄ 0.24 g, distilled water up to a total of 1 L
   ELISA Test Procedure

ELISA test procedure used in this Example was as follows.

First, antigens were diluted in a coating buffer to a concentration of 250 ng/well to aliquot 50 μl into each well of a microtiter plate and coated the well overnight at 4° C. or for 2 hours at 37° C. Then, after the coating solution was discharged, 200 μl aliquots of 1% skim milk were added to each well and blocked at 37° C. for 30 minutes. The resulting product was once washed with TBS-T. Then, 50 μl of hybridoma culture or diluted serum was added to each well and reacted at 37° C. for 2 hours. PBS was used for negative control and serum collected during fusion (1:1000) was used for positive control. After the resulting product was washed 3 times with TBS-T, peroxidase goat anti-mouse IgA+G+M was diluted to 1/5000 to aliquot 50 μl into each well, followed by reaction 37° C. for 1 hour. Next, the resulting product was washed 5 times with TBS-T and 50 μl aliquots of chromophore were added to each well. When the color was changed after about 10 minutes, 100 μl aliquot of 1 N sulfuric acid was added to each well to stop the reaction. Then, O.D at 495 nm was measured for the product.

(2) Immunization of Mouse

Six week-old female BALB/c mice were intraperitoneally injected with 50˜100 ug/0.2 μl of each antigen (KLH-conjugated peptides 4 to 6) mixed with Complete Freund's adjuvant. After four weeks, in the same manner as described above, the mice were intraperitoneally injected with 50˜100 ug/0.2 ml of the above antigen mixed with Incomplete Freund's adjuvant. After two weeks, blood was sampled from the tails of the mice, and it was subjected to an ELISA test. When a threshold of the antibody is from 1/1000 to at least 1.0, the antibody was used in cell fusion. Four weeks after a secondary immune treatment for cell fusion, the same amount of antigen was diluted in PBS and intraperitoneally injected in the mice in an amount of 50˜100 ug/0.2 ml. After 3 days, the mice were sacrificed and cell fusion procedure was performed.

(3) Cell Fusion Procedure

First, myeloma cells purchased from ATCC (Sp2/0-Ag14) were diluted in a 10% FBS medium in a concentration 1×10⁵ cell/ml and incubated overnight at 37° C. in a CO₂ incubator, which was repeated every day for a subculture. On the day prior to cell fusion, 50 ne was subcultured in three 150 T flasks, respectively, for use in cell fusion.

Then, the above immunized mice were sacrificed and spleens were removed from them. Each spleen was washed in a prepared medium (DMEM 10 ml) in a 100 mm dish. Next, a mesh was placed into a 100 mm dish (DMEM 5 ml) and the spleen was disposed on the mesh to be cut into several pieces using scissors. The cut spleen pieces were finely mashed using a plunger portion of a 5 ml syringe, and then spleen cells were separated. The cells were suspended several times using a disposable pipette and the spleen cells were layered in a tube containing previously prepared FBS. The cells were left for about 10 minutes to precipitate large sediments. The cells in an upper portion were uniformly dispersed and suspended in 10 ml of RBC lysate buffer previously warmed, which was incubated at 37° C. for 10 minutes. 3 ml of FBS was added to the culture and the culture was then centrifuged at 1,000 rpm for 10 minutes. After the centrifugation, a supernatant was discarded and a pellet was diluted in a base medium, which was centrifuged at 1,000 rpm for 10 minutes. The centrifuged cells were diluted in 10 ml of a base medium to count the cells.

The above myeloma cells (Sp2/o-Ag14, ATCC) were grown in three 150 T flasks and transferred to 50 ml conical tubes for centrifugation. The pellet in each tube was collected and diluted in 20 ml of a base medium to count the cells.

The above prepared spleen cells and myeloma cells were mixed in a ratio of 5:1 and centrifuged at 1,000 rpm for 10 minutes. The pellet was evenly spread at the end of the tube so as not to form a mass, and 1 ml of PEG(1,500) (based on 1×10⁸ cell) which had been previously warmed was dropped to the cells while slowly rotating the tube for one minute so that the PEG infiltrated through the cells. Again, the tube was slowly rotated for one minute so that the PEG infiltrated through the cells. At this time, a care was taken so that the rotation time did not lapse two minutes in total, and the temperature was maintained at 37° C. 4 ml of a base medium was slowly added to the tube for 4 minutes while tapping the tube, this process being repeated once more. 3 ml of FBS was added to the tube and centrifuged at 1,000 rpm for 10 minutes. The pellet was carefully suspended with 1×HAT medium to aliquiot 200 μl into a 96 well-plate.

Cloning of Hybridoma Cells

The ELISA test was performed for the reduced and oxidized forms of the above peptides which were conjugated to BSA. Cell lines which only responded positively to the oxidized form of peptide in the ELISA test (cell lines in positive wells) were transferred to a 24-well plate and the cells were counted. The cells were diluted in 20 ml of a HT medium to a concentration of 100 to 200 cell/20 ml to aliquot 200 ml into a 96-well plate. Next, the cells were incubated at 37° C. in a CO₂ constant temperature incubator for about 7 to 10 days and feeding was effected once on about 5^{th} to 7^{th} day. Screening by ELISA and cloning procedure were repeated to obtain a final clone. The obtained clone was stored in a liquid nitrogen tank.

Thus, hybridoma cells were obtained which produce monoclonal antibodies specific to peptides 4 to 6, respectively. A hybridoma cell which produces a monoclonal antibody (clone 2A1) specific to peptide 4 was named LF-KSW 4 and deposited with the Korean Cell Line Research Foundation (KCLF), International Depository Authority, on Aug. 22, 2003 (Accession No. KCLRF-BP-00088). A hybridoma cell which produces a monoclonal antibody specific to peptide 4 (clone 10A1) was named LF-KSW 5 and deposited with KCLF on Jun. 17, 2004 (Accession No. KCLRF-BP-00098). A hybridoma cell which produces a monoclonal antibody specific to peptide 5 (clone 2G5) was named LF-KSW 6 and deposited with KCLF on Jun. 17, 2004 (Accession No. KCLRF-BP-00099). A hybridoma cell which produces a monoclonal antibody specific to peptide 6 (clone 4A1) was named LF-KSW 7 and deposited with KCLF on Jun. 17, 2004 (Accession No. KCLRF-BP-000100).

(4) Determination of Isotype of Monoclonal Antibody

Isotype of the monoclonal antibody produced by each hybridoma cell obtained in the above procedure was determined using a subisotyping kit available from Pierce adopting the ELISA sandwich method.

First, antigens were diluted in a coating buffer to a concentration of 250 ng/well to aliquot 50 μl into each well of a microtiter plate and coated the well overnight at 4° C. or for 2 hours at 37° C. Then, after the coating solution was discharged, 200 μl aliquots of 1% skim milk were added to each well and blocked at 37° C. for 30 minutes. The resulting product was once washed with TBS-T. Then, 50 μl of hybridoma cell culture was added to relevant wells (9 wells) and reacted at 37° C. for 2 hours. After the resulting product was washed 3 times with TBS-T, IgM, IgG1, IgG2a, IgG2b, IgG3, and IgA were respectively introduced into each well to be tested and reacted at 37° C. for 1 hour, with normal rabbit IgG being used as a control. After the resulting product was washed 3 times with TBS-T, goat anti-rabbit IgG peroxidase conjugate was diluted to 1/5000 to aliquot 50 μl into each well and reacted at 37° C. for 1 hour. Next, the resulting product was washed five times with TBS-T and 50 μl aliquots of substrate-chromophore were added to each well. When the color was altered, O.D at 495 nm was measured after about 10 minutes.

As a result, it was confirmed that antibodies to peptide 4 (clone 2A1 and clone 10A1), an antibody to peptide 5 (clone 2G5), and an antibody to peptide 6 (clone 4A1) are all IgG1 isotypes.

(5) Confirmation of Specificity of Monoclonal Antibody Using Western Blotting

Western blotting was performed in the same manner as in Example 3 except that monoclonal antibodies (clones 2A1, 10A1, 2G5, and 4A1) were respectively used instead of the polyclonal antibody, to confirm the specificity of the monoclonal antibodies.

The results are shown in FIGS. 8 through 11. The upper portion of FIG. 8 is a view illustrating results of western blotting of HeLa cell lysate not treated with H₂O₂ (lane 1), HeLa cell lysate treated with H₂O₂ (lane 2), Prx I in a reduced state (lane 3), and Prx I in an oxidized state (lane 4), detected using HRP-anti SO₃-Prx I (clone 2A1). The lower portion of FIG. 8 is a view illustrating results of performing again western blotting of the above membrane, detected using HRP-anti-Prx I.

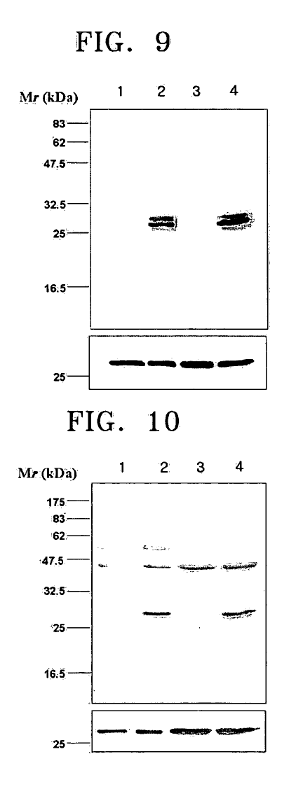

The upper portion of FIG. 9 is a view illustrating results of western blotting of 20 ug of HeLa cell lysate not treated with H₂O₂ (lane 1), 20 ug of HeLa cell lysate treated with H₂O₂ (lane 2), 40 ug of HeLa cell lysate not treated with H₂O₂ (lane 3), and 40 ug of HeLa cell lysate treated with H₂O₂ (lane 4), detected using HRP-anti SO₃-Prx I (clone 10A1). The lower portion of FIG. 9 is a view illustrating results of performing again western blotting of the above membrane, detected using HRP-anti-Prx I.

The upper portion of FIG. 10 is a view illustrating results of western blotting of 20 ug of HeLa cell lysate not treated with H₂O₂ (lane 1), 20 ug of HeLa cell lysate treated with H₂O₂ (lane 2), 40 ug of HeLa cell lysate not treated with H₂O₂ (lane 3), and 40 ug of HeLa cell lysate treated with H₂O₂ (lane 4), detected using HRP-anti SO₃-Prx VI (clone 2G5). The lower portion of FIG. 10 is a view illustrating results of performing again western blotting of the above membrane, detected using HRP-anti-Prx VI.

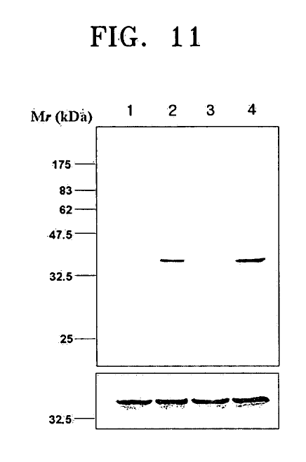

The upper portion of FIG. 11 is a view illustrating results of western blotting of 20 ug of HeLa cell lysate not treated with H₂O₂ (lane 1), 20 ug of HeLa cell lysate treated with H₂O₂ (lane 2), 40 ug of HeLa cell lysate not treated with H₂O₂ (lane 3), and 40 ug of HeLa cell lysate treated with H₂O₂ (lane 4), detected using HRP-anti SO₃-GAPDH (clone 4A1). The lower portion of FIG. 11 is a view illustrating results of performing again western blotting of the above membrane, detected using HRP-anti-GAPDH.

According to an embodiment of the present invention, an antibody specific to a sulfonylated protein isofom can be efficiently produced.

The antibody specifically binding to a sulfonylated protein isoform can be effective in an analytical method for determining whether cells were under oxidative stress using antibodies or an analytical method for determining whether a protein containing cystine at its active site lost its activity.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A method for producing an antibody binding only to a sulfonylated isoform of a protein; not to a non-sulfonylated isoform of the protein and other proteins, comprising:
providing a peptide comprised of 7 to 15 amino acids derived from the protein and having a sulfonylated cysteine residue;
inducing an antibody to the peptide in an animal; and isolating a population of antibodies reactive to the peptide.

2. The method of claim 1, further comprising screening an antibody unreactive to the non-sulfonylated protein isoform having a non-sulfonylated cysteine residue from the isolated population of antibodies, and collecting the screened antibody.

3. The method of claim 1 or 2, further comprising verifying that the antibody obtained from the isolating or the collecting specifically binds to the protein isoform having a sulfonylated cysteine residue; not to other proteins.

4. The method of claim 1, wherein the protein is glyceraldehyde-3-phosphate dehydrogenase (GAPDH), peroxiredoxin I (Prx I) or peroxiredoxin VI (Prx VI).

5. The method of claim 1, wherein in the providing a peptide of 7 to 15 amino acids and having a sulfonylated cysteine residue, the sulfonylated peptide is any one of peptides having amino acid sequences of SEQ ID Nos. 1, 2, and 3 in which cysteins in 6, 6 and 8 positions are respectively sulfonylated.

6. An antibody which specifically binds only to a sulfonylated isoform of a protein having a sulfonylated cysteine residue; not to a non-sulfonylated isoform of the protein and other proteins.

7. The antibody of claim 6, which specifically binds to a sulfonylated Prx I isoform having a sulfonylated Cys 52

8. The antibody of claim 7, which is a monoclonal antibody produced by a hybridoma cell under accession No. KCLRF-BP-00088 or KCLRF-BP-00098.

9. The antibody of claim 6, which specifically binds to a sulfonylated Prx VI isoform having a sulfonylated Cys 47.

10. The antibody of claim 9, which is a monoclonal antibody produced by a hybridoma cell under accession No. KCLRF-BP-00099 (Prx VI).

11. The antibody of claim 6, which specifically binds to a sulfonylated GAPDH isoform having a sulfonylated Cys 152.

12. The antibody of claim 11, which is a monoclonal antibody produced by a hybridoma cell under accession No. KCLRF-BP-000100.

13. The antibody of any one of claims 6 to 12, which is produced using the method of any one of claims 1, 2, 4 and 5.


SEQUENCE LISTING (6 sequences)

SEQ ID NO: 1 - protein, 10 aa, Artificial Sequence
  peptide 1 of Prx I
DFTFVCPTEI

SEQ ID NO: 2 - protein, 10 aa, Artificial Sequence
  peptide 2 of Prx VI
DFTPVCTTEL

SEQ ID NO: 3 - protein, 11 aa, Artificial Sequence
  peptide 3 of GAPDH
KIISNASCTTN

SEQ ID NO: 4 - protein, 10 aa, Artificial Sequence
  peptide 4 of Prx 1 ; a sulfonylated form of peptide 1
DFTFVXPTEI

SEQ ID NO: 5 - protein, 10 aa, Artificial Sequence
  peptide 5 of Prx VI; a sulfonylated form of peptide 2
DFTPVXTTEL

SEQ ID NO: 6 - protein, 11 aa, Artificial Sequence
  peptide 6 of GAPDH ; a sulfonylated form of peptide 3
KIISNASXTTN